(19) United States
(12) Patent Application Publication
(10) Pub. No.: US 2020/0128195 A1
(43) Pub. Date: Apr. 23, 2020

(54) ELECTRONIC MAKEUP MIRROR APPARATUS AND DISPLAY METHOD THEREOF

(71) Applicant: CAL-COMP BIG DATA, INC., NEW TAIPEI CITY (TW)

(72) Inventors: Chia-Ming Yong, New Taipei City (TW); Min-Chang Chi, New Taipei City (TW)

(73) Assignee: CAL-COMP BIG DATA, INC., NEW TAIPEI CITY (TW)

(21) Appl. No.: 16/252,710

(22) Filed: Jan. 21, 2019

(30) Foreign Application Priority Data
Oct. 17, 2018 (CN) 201811208544.1

(51) Int. Cl. H04N 5/272; G06K 9/00; G06K 9/32; G06K 9/20; G01N 33/483

(52) U.S. Cl. CPC H04N 5/272; G06K 9/00255; G01N 33/4833; G06K 9/2027; G06K 9/3233

(57) ABSTRACT

An electronic makeup mirror apparatus and a display method thereof are provided. The electronic makeup mirror apparatus includes a screen, an image capturing device, and a processor. The screen displays a reference region. The screen includes a light source generation module, and the light source generation module provides a first movable light source. The processor is connected to the screen and the image capturing device. The processor determines that whether a face region overlaps with the reference region, obtains an image of the face region through the image capturing device when the face region overlaps with the reference region and controls the light source generation module of the screen to emit a first movable light source.

### CROSS-REFERENCE TO RELATED APPLICATION

This application claims the priority benefit of Chinese application serial no. 201811208544.1, filed on Oct. 17, 2018. The entirety of the above-mentioned patent application is hereby incorporated by reference herein and made a part of this specification.

### BACKGROUND

### Technical Field

The disclosure relates to an electronic makeup mirror apparatus and a display method thereof.

### Description of Related Art

The length or speed of time is a relative concept, but skin aging is not. No matter how a user feels that time goes fast or moves slow, the skin of the user ages as seasons rotate in an unnoticeable way. Nevertheless, along with technology advancement, skin condition and the degree of aging may be evaluated through image analyses.

When skin condition and the degree of aging of the user are evaluated, image analyses are required to be performed, face features are required to be obtained, each of the features is required to be analyzed, and skin condition and the degree of aging are required to be evaluated, so the user has to spend time waiting for all these processes to be completed. How to allow the user to feel less anxious and enjoy enhanced technological experience when waiting for the analysis is an important issue in this field.

### SUMMARY

The disclosure provides an electronic makeup mirror apparatus and a display method thereof capable of providing a novel analysis image in a face analysis process to reduce anxiety of a user when waiting.

An embodiment of the disclosure provides an electronic makeup mirror apparatus including a display screen, an image capturing device, and a processor. The display screen displays a reference region and includes a light source generation module. The light source generation module provides a first movable light source. The processor is connected to the display screen and the image capturing device. In response to that a face region is determined to overlap with the reference region through the image capturing device, the processor controls the image capturing device to obtain the face region and controls the light source generation module of the screen to emit a first movable light source.

An embodiment of the disclosure further provides a display method applicable to an electronic makeup mirror apparatus, and the display method includes the following steps. A reference region is displayed. Whether a face region overlaps with the reference region is determined. In response to that, the face region is determined to overlap with the reference region, the face region is obtained and a first movable light source is emitted.

To sum up, in the electronic makeup mirror apparatus and the display method thereof for performing face analysis, the anxious feeling of the user is shifted when the user waits for the analysis of his/her skin texture as the movable light source is controlled, and the use may also enjoy an enhanced technological experience.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram illustrating a system of an electronic makeup mirror apparatus according to an exemplary embodiment of the disclosure.

FIG. 2 is a schematic diagram illustrating circuit connection of the electronic makeup mirror apparatus according to an exemplary embodiment of the disclosure.

FIG. 3 is a schematic flow chart illustrating a display method according to an exemplary embodiment of the disclosure.

FIG. 4A to FIG. 4F are schematic views illustrating movement of a first movable light source in the electronic makeup mirror apparatus.

FIG. 5 is a schematic view illustrating movable light sources of the electronic makeup mirror apparatus according to another exemplary embodiment of the disclosure.

FIG. 6 is a schematic view illustrating display content of the electronic makeup mirror apparatus according to an exemplary embodiment of the disclosure.

FIG. 7 is a schematic view illustrating the display content of the electronic makeup mirror apparatus according to an exemplary embodiment of the disclosure.

### DESCRIPTION OF THE EMBODIMENTS

Descriptions of the disclosure are given with reference to the exemplary embodiments illustrated by the accompanying drawings. Wherever possible, the same reference numbers are used in the drawings and the description to refer to the same or like parts.

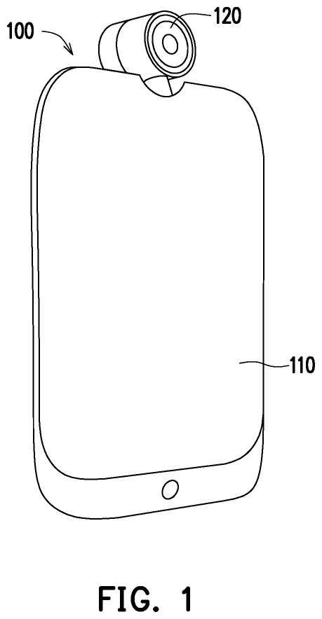

FIG. 1 is a schematic diagram illustrating a system of an electronic makeup mirror apparatus according to an exemplary embodiment of the disclosure. With reference to FIG. 1, an electronic makeup mirror apparatus 100 has a screen 110 and an image capturing device 120.

The screen 110 displays a current image and a variety of messages provided by the electronic makeup mirror apparatus 100. In an embodiment of the disclosure, the screen 110 is composed of a surface mirror and a display screen. The surface mirror is made of a highly-reflective material (e.g., a mirror) and is configured to display the current image, for example, an image of a user and a background image of the user and the like, through light refraction. The display screen is disposed behind the surface mirror and is configured to display the messages. For instance, the display screen may be a liquid crystal display (LCD), an organic light-emitting diode (OLED) display, a thin film transistor liquid crystal display (TFT-LCD), etc., and the disclosure is not limited in this regard. In an embodiment of the disclosure, the display screen may also be a touch display screen of a variety of types, such as a capacitive touch display screen, a resistive touch display screen, etc.

Since the surface mirror is made of a highly-reflective material, when the display screen does not display the backlight, the surface mirror reflects the current image (e.g., a facial image of the user and the background image or the background image). If the display screen displays a high brightness light ray, the light ray may penetrate the surface mirror and reaches the eyes of the user, so that the user can see the messages displayed on the display screen. As such, through the combination of the surface mirror and the display screen, the user may see himself/herself through the surface mirror and browses messages at the same time.

The image capturing device 120 is configured to obtain images. The image capturing device 120 is, for example, a video camera or a camera embedded in the electronic makeup mirror apparatus 100 or a cell phone, camera, etc. externally connected to the electronic makeup mirror apparatus, and the disclosure is not limited in this regard.

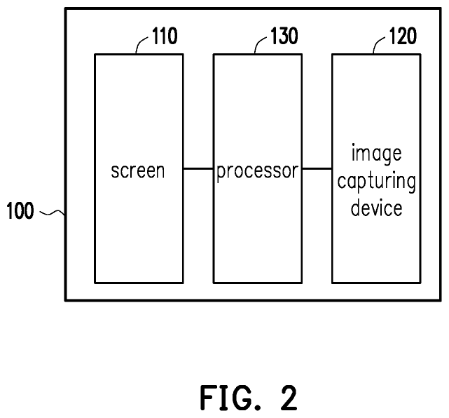

FIG. 2 is a schematic diagram illustrating circuit connection of the electronic makeup mirror apparatus according to an exemplary embodiment of the disclosure. With reference to FIG. 2, the screen 110 of this embodiment further includes a light source generation module 112. The light source generation module 112 consists of a controller (e.g., a light source controller chip) and light source emission devices (e.g., light-emitting diodes, cold cathode lamp tubes, etc.). The controller is configured receive a display control signal and accordingly controls switches of the light source emission devices to emit light sources. That is, the screen 110 displays a variety of images on the screen 110 through the light sources provided by the light source generation module 112.

The light sources provided by the light source generation module 112 feature identical or different light source features. The light source features may include, for example, a brightness value, a color value, a contrast value, or a combination of the foregoing parameters or may be any light source features capable of displaying different visual effects, and theses light source features may all be applied in the disclosure. In an exemplary embodiment, in a first light source feature, the brightness value may range between, for example, 0 and 100, and the color value may be adjusted based on the respective three primary colors of light. That is, the red value may range between 0 and 255, the blue value may range between 0 and 255, and the yellow value may range between 0 and 255. Nevertheless, the values of the ranges of the light source features may be different owing to parameters designed for the screen 110 and the light source generation module 112, and the disclosure is not limited in this regard.

In this embodiment, the light source generation module 112 provides a movable light source. Specifically, as regards the movable light source, the controller controls turning on/off states of the light source emission devices corresponding to different regions of the screen 110 according to the display control signal, so that a viewer may see that the light source is moving. Nevertheless, in other embodiments, a movable light source emission device may also be disposed in the light source generation module 112, and the disclosure is not limited in this regard.

The electronic makeup mirror apparatus 100 of this embodiment further includes a processor 130. The processor 130 is coupled to the screen 110 and the image capturing device 120 and performs a variety of operations when the electronic makeup mirror apparatus 100 works. In addition, in this embodiment, the processor 130 further processes images and/or messages and then provides a display signal to the light source generation module 112 according to an image processing result or a message processing result. The processor 130 may be, for example, a central processing unit (CPU), a microprocessor, an application specific integrated circuit (ASIC), a programmable logic device (PLD), or other similar devices.

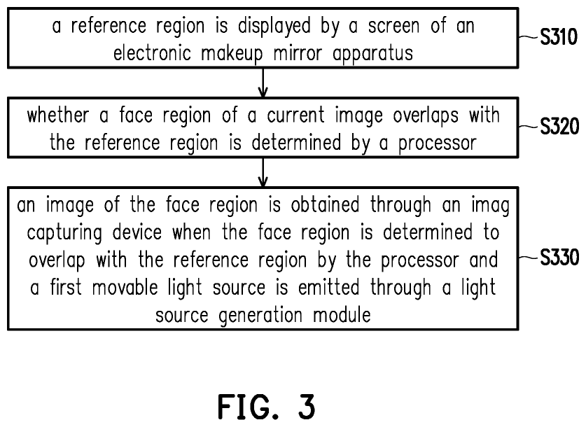

FIG. 3 is a schematic flow chart illustrating a display method according to an exemplary embodiment of the disclosure. In this embodiment, the display method is at least suited to the electronic makeup mirror apparatus 100 of FIG. 1, and the flow chart representing implementation of the display method is described through FIG. 1 and FIG. 3.

The display method is suited to be used when the user activates the face scanning function of the electronic makeup mirror apparatus 100, for example, when the user activates scenarios such as skin testing, face analyzing, etc. First, in step S310, a reference region is displayed by the screen 110 of the electronic makeup mirror apparatus 100.

Specifically, an effect of face analysis testing is to be affected by the size and position of the face of the user. Therefore, in the embodiments of the disclosure, the processor 130 displays the reference region in the screen 110 through the light source generation module 112, so as to prompt the user to position the face to fit in the reference region. The reference region may be any pattern, for example, a circle, a quadrangle, a pentagon, a hexagon, etc., which can prompt the user to match the position of the face with the reference region.

In this embodiment, the reference region is a pattern corresponding to the face region. Specifically, the reference region includes a facial reference contour and a facial feature contour. The facial reference contour is configured to prompt the user to position the face to fit in the facial reference contour and position the facial features to fit in the facial feature contour. The reference region may be adjusted according to different model numbers of the electronic makeup mirror apparatus 100, and the disclosure does not intend to limit the position of the reference region. Note that the screen 110 controls the light source generation module 112 to display a reference light source at a position relative to the facial reference contour and the facial feature contour so as to display the reference region. The reference light source has a first light source feature. For instance, the first light source feature may feature, for example, a brightness value of 68, a red value of 255, a yellow value of 255, and a blue value of 255.

In step S320, the processor 130 determines that whether the face region overlaps with the reference region. To be specific, the overlapping described in the disclosure is that the face region displayed by the screen 110 is located in the reference region, so that the face region and the reference region are visually overlapped.

The method used by the processor 130 to determine whether the face region and the reference region are overlapped is, for example, setting an image obtained by the image capturing device 120 to correspond to a pixel range of the screen 110 by the processor 130. As such, the processor 130 determines that whether a position of the face region corresponding to the screen 110 is identical to a position of the reference region through the image capturing device 120, but the disclosure is not limited in this regard.

Note that since face shapes and sizes and positions of the facial features of people are all different, overlapping is determined by the processor 130 when the facial features of a user all fit in the facial feature contour in an embodiment of the disclosure. Alternatively, overlapping may also be determined by the processor 130 according to an overlapping ratio of the face region and the reference region, and the disclosure is not limited in this regard.

In step S330, when determining that the face region overlaps with the reference region, the processor 130 obtains an image of the face region through the image capturing device 120 and emits a first movable light source through the light source generation module 112. At the same time, the processor 130 activates a skin testing procedure, so as to perform skin testing and face analyzing according to the image obtained by the image capturing device 120.

In an embodiment of disclosure, the processor 130 controls the first movable light source to move in a first direction through the light source generation module 112. The first direction is, for example, a vertical direction, a horizontal direction, a diagonal direction, an inward-facing direction from an edge, an outward-facing direction from a center, a helical direction, etc. along the screen 110 or may be a direction defined by the user himself/herself, and the disclosure is not limited thereto.

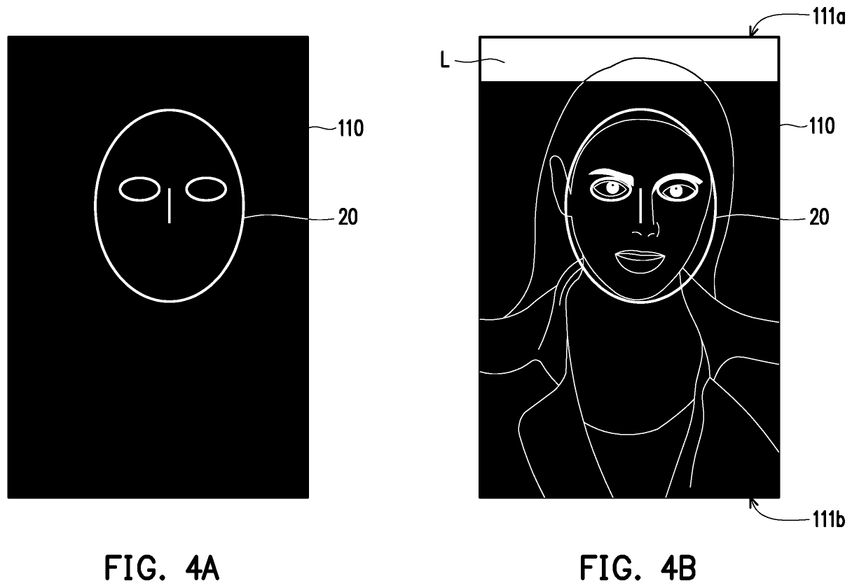

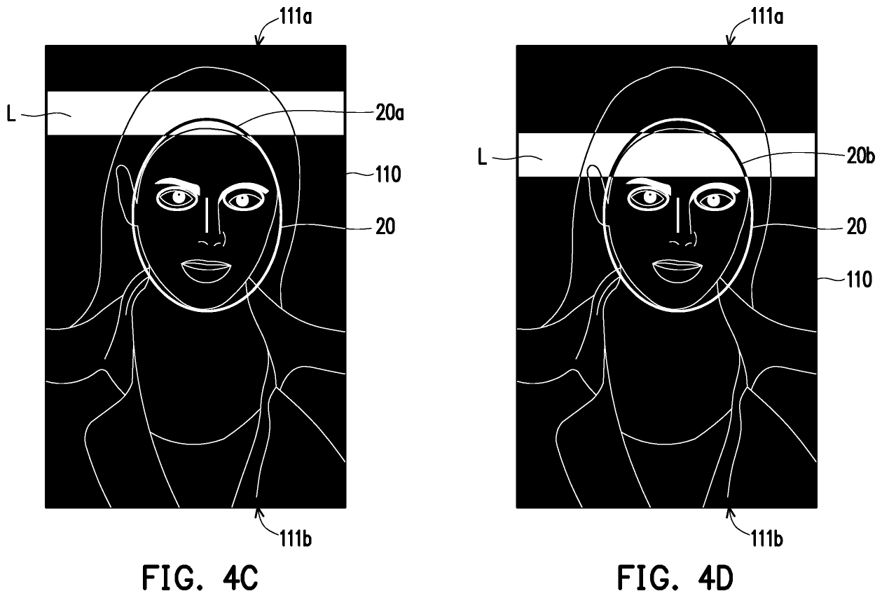

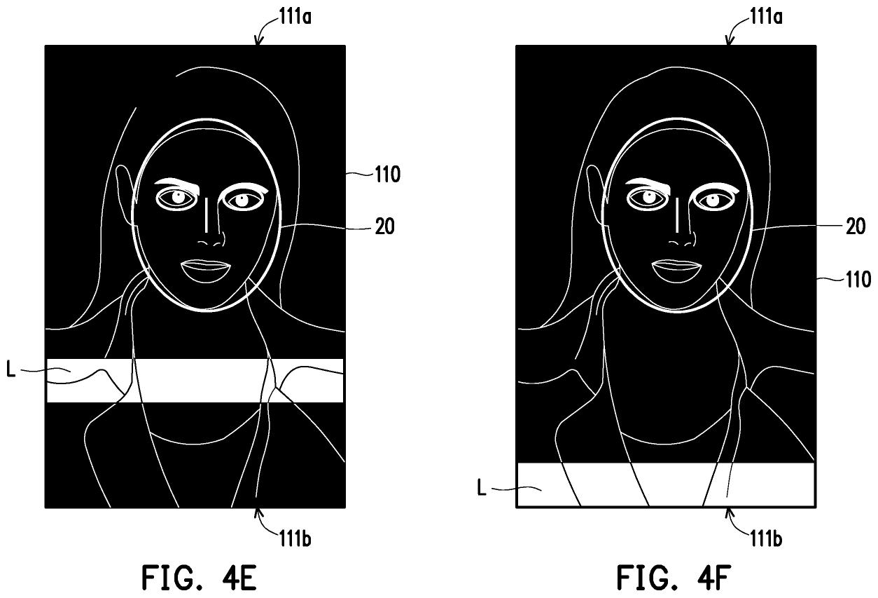

Movement of the first movable light source is described through FIG. 4A to FIG. 4F. With reference to FIG. 4A to FIG. 4F, FIG. 4A to FIG. 4F are schematic views illustrating movement of the first movable light source in the electronic makeup mirror apparatus. In the following embodiment, the first direction is a vertical direction along the screen 110 and is a direction vertically moving from a first side 111a of the screen 110 to a second side 111b.

In FIG. 4A, the screen 110 displays a reference region 20 to prompt the user to position the face region to fit in the reference region 20.

In FIG. 4B, the processor 130 determines that the face region of the user fits in the reference region and provides a first movable light source L located at the first side 111a of the screen 110.

In FIG. 4C, at this time, the processor 130 controls the light source generation module 112 and vertically moves the first movable light source L to a lower portion of the screen 110.

In this embodiment, at the same time, a reference light source of the reference region 20 is switched from the first light source feature to a second light source feature. As described above, the first light source feature may feature, for example, a brightness value of 68, a red value of 255, a yellow value of 255, and a blue value of 255. The switched second light source feature may feature, for example, a brightness value of 68, a red value of 0, a yellow value of 0, and a blue value of 255.

In this embodiment, not only the reference light source of the reference region 20 is switched from the first light source feature to the second light source feature, but also a reference light source of a portion 20a of the reference region correspondingly overlapping with the first movable light source L is switched from the second light source feature to a third light source feature featuring, for example, a brightness value of 68, a red value of 0, a yellow value of 255, and a blue value of 255. Note that in an embodiment of the disclosure, the third light source feature is different from the first light source feature, so that the user may enjoy different visual experiences.

In addition, in this embodiment, when the first movable light source L and the reference region 20 are overlapped, the processor 130 also controls the light source generation module 112 to lower a moving speed of the first movable light source L. Nevertheless, the disclosure is not limited in this regard.

Note that the processor 130 determines that whether the first movable light source L overlaps with the reference region 20 according to the position of the first movable light source L emitted by the light source generation module 112 and the position of the reference region 20 in the screen 110, but the disclosure is not limited thereto.

In FIG. 4D, the first movable light source L continues to move downward and overlaps with a portion 20b of the reference region. At this time, the portion 20a of the reference region overlapping with the first movable light source L in FIG. 4C is changed to not to overlap with the first movable light source L. At this time, the portion 20a of the reference region is switched from the third light source feature back to the second light source feature. The portion 20b of the reference region overlapping with the first movable light source L is switched from the second light source feature to the third light source feature, and repeated description is omitted herein.

In FIG. 4E, the first movable light source L continues to move downward and no longer overlaps with the reference region 20. At this time, the reference region 20 is switched from the second light source feature to the first light source feature. That is, in this embodiment, when the first movable light source L does not overlap with the reference region 20, the reference region 20 displays the first light source feature. When the first movable light source L overlaps with the reference region 20, the reference region 20 displays the second light source feature, and the portion of the reference region overlapping with the first movable light source L displays the third light source feature.

In FIG. 4F, the first movable light source L continues to move downward to the second side 111b of the screen 110. At this time, the reference light source of the reference region 20 is switched from the first light source feature or the second light source feature to a fifth light source feature featuring, for example, a brightness value of 100, a red value of 255, a yellow value of 255, and a blue value of 255. In an embodiment of the disclosure, after moving from the first side 111a of the screen 110 to the second side 111b of the screen 110, the first movable light source L once again moves from the second side 111b of the screen 110 back to the first side 111a of the screen 110. At this time, the process of how the reference region 20 and the first movable light source L are displayed on the screen 110 is identical that shown in FIG. 4A to FIG. 4F, and a difference therebetween includes only the change of the directions, so that repeated description is omitted herein.

In another embodiment of the disclosure, in FIG. 4C and FIG. 4D, the reference light source of the reference region 20 is not switched from the first light source feature to the second light source feature. Nevertheless, the reference light source of the portion 20a of the reference region or the portion 20b of the reference region correspondingly overlapping with the first movable light source L is directly switched from the first light source feature to the third light source feature featuring, for example, a brightness value of 68, a red value of 0, a yellow value of 255, and a blue value of 255. Further, when the first movable light source L keeps moving so that the portion 20a of the reference region or the portion 20b of the reference region no longer overlaps with the first movable light source L, the portion 20a of the reference region or the portion 20b of the reference region is switched from the third light source feature back to the first light source feature.

Note that if the user moves during the skin testing procedure so that the face region no longer overlaps with the reference region and that the skin testing procedure is not completed, at this time, the processor 130 controls the reference light source of the reference region 20 to be switched from the second light source feature to a fourth light source feature featuring, for example, a brightness value of 100, a red value of 255, a yellow value of 0, and a blue value of 0. In this way, the user is prompted that the skin testing procedure is not completed.

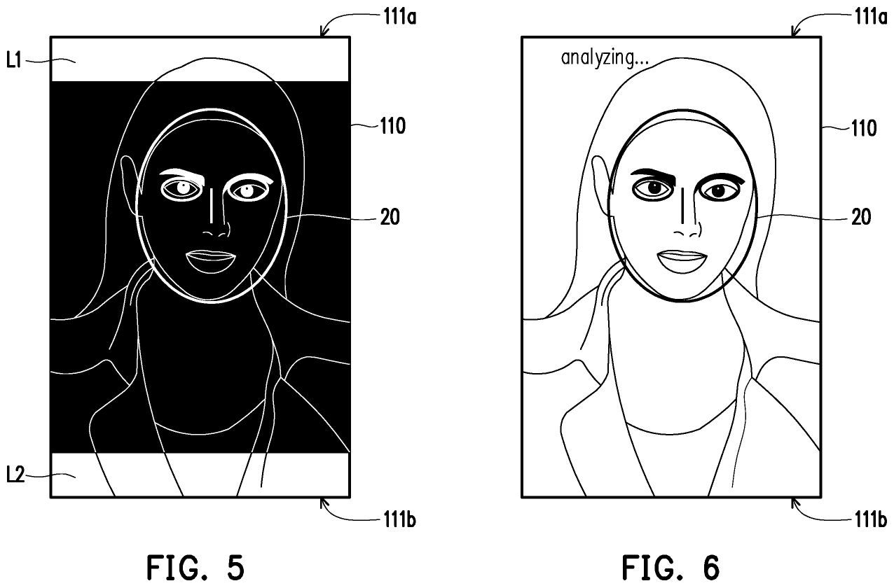

FIG. 5 is a schematic view illustrating movable light sources of the electronic makeup mirror apparatus according to another exemplary embodiment of the disclosure. With reference to FIG. 5, in an embodiment of the disclosure, the electronic makeup mirror apparatus 100 not only controls the light source generation module 112 to generate the first movable light source L1 but also controls the light source generation module 112 to generate a second movable light source L2. The first movable light source L1 moves in the first direction, and the second movable light source L2 moves in a second direction. The first direction and the second direction are different or are opposite to each other. The first direction and the second direction are, for example, vertical directions, horizontal directions, diagonal directions, inward-facing directions from edges, outward-facing direction from a center, helical directions, etc. along the screen 110 or may be directions defined by the user himself/herself, and the disclosure is not limited thereto.

In the embodiment shown by FIG. 5, the first movable light source L1 is located at the first side 111a of the screen 110, and the first movable light source L1 vertically moves downward towards the second side 111b. The second movable light source L2 is located at the second side 111b of the screen 110 and moves towards the first side 111a. The first movable light source L1 and the second movable light source L2 move in a manner similar to that shown in FIG. 4A to FIG. 4F, and that repeated description is omitted herein.

With reference to FIG. 6, FIG. 6 is a schematic view illustrating display content of the electronic makeup mirror apparatus according to an exemplary embodiment of the disclosure.

With reference to FIG. 6, after scanning is completed, the electronic makeup mirror apparatus displays a preset processing prompting icon on the screen 110, so as to prompt the user that the electronic makeup mirror apparatus 100 is processing an analysis result.

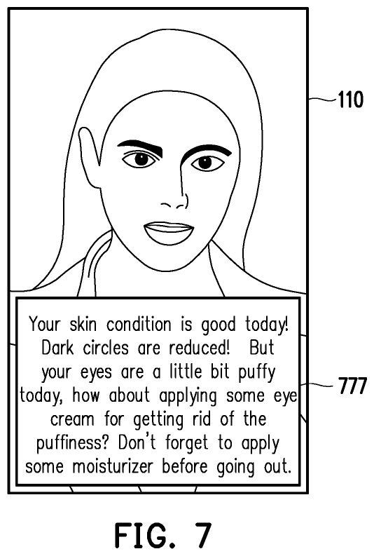

FIG. 7 is a schematic view illustrating the display content of the electronic makeup mirror apparatus according to an exemplary embodiment of the disclosure. In FIG. 7, a facial analysis result 777 is displayed through the screen 110 for providing a reference to the user.

In view of the foregoing, in the electronic makeup mirror apparatus and the display method thereof for performing face analysis, the anxious feeling of the user is shifted when the user waits for the analysis of his/her skin texture as the movable light source is controlled, and the use may also enjoy an enhanced technological experience.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An electronic makeup mirror apparatus, wherein the electronic makeup mirror apparatus comprises:
a screen, displaying a reference region, wherein the screen comprises a light source generation module, and the light source generation module provides a first movable light source;
an image capturing device; and
a processor, connected to the screen and the image capturing device, wherein
the processor determines that whether a face region of a current image overlaps with the reference region, obtains an image of the face region through the image capturing device in response to that the face region is determined to overlap with the reference region, and controls the first movable light source emitted by the light source generation module of the screen.

2. The electronic makeup mirror apparatus as claimed in claim 1, wherein the light source generation module provides a reference light source corresponding to the reference region, and the reference light source comprises a first light source feature.

3. The electronic makeup mirror apparatus as claimed in claim 2, wherein the processor further controls the light source generation module to switch the reference light source from the first light source feature to a second light source feature in response to that the first movable light source overlaps with the reference region.

4. The electronic makeup mirror apparatus as claimed in claim 2, wherein
the processor controls the light source generation module to enable a portion of the reference light source corresponding to the reference region to be switched from the first light source feature to a third light source feature in response to that the first movable light source and the reference region are detected to partially overlap,
the processor controls the light source generation module to switch the portion of the reference light source corresponding to the reference region from the third light source feature back to the first light source feature in response to that the first movable light source overlapping with the portion of reference region is changed to not to overlap with the reference region, wherein at least a brightness value or a color of the third light source feature is different from that of the first light source feature.

5. The electronic makeup mirror apparatus as claimed in claim 2, wherein
the processor activates a skin testing procedure after controlling the light source generation module to emit the first movable light source, the processor determines that whether the skin testing procedure is completed in response to that the image capturing device detects that the face region does not overlap with the reference region, and the processor further controls the light source generation module to stop moving the first movable light source in response to that the skin testing procedure is detected as not completed and controls the light source generation module to switch the reference light source from the first light source feature or the second light source feature to a fourth light source feature.

6. The electronic makeup mirror apparatus as claimed in claim 1, wherein the processor controls the light source generation module to lower a moving speed of the first movable light source in response to that the first movable light source and the reference region overlap.

7. The electronic makeup mirror apparatus as claimed in claim 1, wherein
the processor controls the light source generation module to move the first movable light source in a first direction.

8. The electronic makeup mirror apparatus as claimed in claim 7, wherein
the processor further controls the light source generation module to provides a second movable light source, and the second movable light source is moved in a second direction, wherein the first direction and the second direction are vertical directions, horizontal directions, or diagonal directions of the screen, and the first direction and the second direction are different, or the first direction and the second direction are opposite directions.

9. The electronic makeup mirror apparatus as claimed in claim 1, further comprising:
a surface mirror, reflecting the current image,
wherein the screen does not display the current image.

10. A display method, applicable to an electronic makeup mirror apparatus, wherein the display method comprises:
displaying a reference region;
determining whether a face region overlaps with the reference region; and
obtaining an image of the face region and generating a first movable light source in response to that the face region is determined to overlap with the reference region.

11. The display method as claimed in claim 10, further comprising:
providing a reference light source corresponding to the reference region to display the reference region, and the reference light source comprising a first light source feature.

12. The display method as claimed in claim 11, further comprising:
switching the reference light source from the first light source feature to a second light source feature in response to that the first movable light source overlaps with the reference region.

13. The display method as claimed in claim 12, further comprising:
controlling a portion of the reference light source corresponding to the reference region to be switched from the second light source feature to a third light source feature in response to that the first movable light source and the reference region are detected to be partially overlap; and
controlling the light source generation module to switch the portion of the reference light source corresponding to the reference region from the third light source feature back to the first light source feature in response to that the first movable light source overlapping with the portion of the reference region is changed to not to overlap with the reference region,
wherein at least a brightness value or a color of the third light source feature is different from that of the second light source feature.

14. The display method as claimed in claim 11, further comprising:
activating a skin testing procedure after controlling the light source generation module to emit the first movable light source;
determining that whether the skin testing procedure is completed in response to that detecting that the face region does not overlap with the reference region; and
stopping moving the first movable light source in response to that the skin testing procedure is detected to not completed and controlling the light source generation module to switch the reference light source to be switched from the second light source feature to a fourth light source feature.

15. The display method as claimed in claim 11, further comprising:
lowering a moving speed of the first movable light source in response to that the first movable light source and the reference region overlap.

16. The display method as claimed in claim 10, further comprising:
controlling the first movable light source to be moved in a first direction.

17. The display method as claimed in claim 16, further comprising:
providing a second movable light source, and the second movable light source is moved in a second direction,
wherein the first direction and the second direction are vertical directions, horizontal directions, or diagonal directions corresponding to the screen of the electronic makeup mirror apparatus, and the first direction and the second direction are different, or the first direction and the second direction are opposite directions.

18. The display method as claimed in claim 16, further comprising:
controlling the reference light source to be switched from the second light source feature to a fifth light source feature in response to that the first movable light source is moved from a first side of the screen to a second side in the first direction and the first movable light source moves to the second side.

19. The display method as claimed in claim 10, wherein the reference region corresponds to a facial contour or a geometric pattern.
